(19) United States
(12) Patent Application Publication
(10) Pub. No.: US 2001/0049350 A1
(43) Pub. Date: Dec. 6, 2001

(54) GROWTH INHIBITION AND ERADICATION OF SOLID TUMORS USING NEUROENDOCRINE RESETTING THERAPY AND PHOTODYNAMIC THERAPY

(76) Inventors: ANTHONY H. CINCOTTA, CHARLESTOWN, MA (US); LOUIS CINCOTTA, ANDOVER, MA (US)

Correspondence Address: DARBY & DARBY, 805 THIRD AVENUE, NEW YORK, NY, 10022

(21) Appl. No.: 09/187,768

(22) Filed: Nov. 6, 1998

Related U.S. Application Data
(63) Continuation of application No. 08/838,079, filed on Apr. 15, 1997, now abandoned.
(60) Provisional application No. 60/016,619, filed on May 1, 1996.

(51) Int. Cl. A61K 38/00; G01N 33/48

(52) U.S. Cl. 514/2; 436/64

(57) ABSTRACT

A method of ablating the growth of or eradicating tumors in mammals having prolactin, growth hormone, and melatonin daily rhythms by adjusting one or more of the prolactin, growth hormone, and melatonin profiles of the mammal to conform to or approach the corresponding normal profile for healthy members of the same species and sex as said mammal, contacting the cells of the tumor with a photoactive photosensitizer, and, exposing the photosensitizer-contacted tumor cells to light of a predetermined wavelength, power density, and energy level.

### BACKGROUND OF THE INVENTION

### Field of the Invention

Disclosed herein are methods for treating tumors. More specifically, this invention pertains to methods for arresting the growth of or eradicating tumors in mammals having prolactin and melatonin daily rhythms and in need of such treatment by

(a) adjusting one or both of the prolactin and melatonin profiles of the mammal to conform to or approach the respective standard profile for healthy members of the same species and sex as said mammal,

(b) contacting the cells of the tumor with a photosensitizer, and

(c) exposing the contacted tumor cells to light of a predetermined wavelength, power density, and energy level.

Treatment of Tumors

It is well known in the art to inhibit the growth of tumors by using cytotoxic compositions or ionizing radiation. A major drawback of ionizing radiation as a therapeutic modality is that it often results in injury or damage to healthy tissue in the vicinity of, or in contact with, the malignant tumor cells. Cytotoxic compositions have the even greater drawback of often causing systemic toxicity, i.e. damaging tissues at loci distal to the tumor.

One known method for killing or treating tumor cells is by contacting them with a photosensitizer substance and thereafter exposing the contacted cells to light of a predetermined wavelength (Kessel, D., International Photodynamics March 1995, pp. 2-3; Dougherty, T. J. et al., Photochem. Photobiol. 45:879-89, 1987; Moan, J. et al., Photochem. Photobiol. 55:145-57, 1992). This so-called photodynamic therapy (PDT) for treatment of tumors selectively eradicates tumor tissue without the deleterious side effects that are often seen when ionizing radiation or chemotherapy is employed. However, the two most widely studied PDT drugs, hematoporphyrin (HPD) and Photofrin II, suffer from several limitations such as:

(i) both exhibit a low absorption coefficient in the region where light penetrates tissue most efficiently (600-800 nm);

(ii) both are complex mixtures of porphyrin ethers and ester oligomers;

(iii) prolonged retention of these photosensitizers in the skin leads to dermal photosensitization that can persist for months.

It has now been unexpectedly discovered that the efficacy of photodynamic therapy for arresting the growth of or eradicating tumors can be Significantly enhanced by normalizing the prolactin and/or melatonin profiles of the mammal receiving such treatment to approach or conform to the respective profiles of a young, healthy mammal of the same sex and species.

Prolactin and Neuroendocrine Rhythms

Research has demonstrated that circadian rhythms play important roles in regulating prolactin activities and vice versa.

Publications such as Meier, A. H., Gen. Comp. Endocrinol. 3(Suppl 1):488-508, 1972; Meier, A. H., Trans. Am. Fish. Soc. 113:422431, 1984; Meier, A. H. et al., Current Ornithology II (ed Johnston R. E.) 303-343, 1984; Cincotta, A. H. et al., J. Endocrinol. 120:385-391, 1989; Meier, A. H., Amer. Zool. 15:905-916, 1975; Meier, A. H., Hormonal Correlates of Behavior (eds. Eleftherton and Sprott) 469-549, 1975 disclose how circadian rhythms regulate prolactin activities. The resulting daily variations in responsiveness of different cell types to prolactin have a primary role in regulating numerous physiological processes, including fat storage, lipogenic responsiveness to insulin, migratory behavior, metamorphosis, reproduction, growth, pigeon cropsac development and mammary development (Meier, A. H., Gen. Comp. Endocrinol. 3(Suppl 1):488-508, 1972; Meier, A. H., Amer. Zool. 15:905-916, 1975; Meier, A. H. et al., Science 173:1240-1242, 1971). In regulating one of the foregoing physiological activities, prolactin may be observed to produce a stimulatory or an inhibitory effect on a given activity, or to have no effect on it. These varying effects have recently been shown in animals to be a function of the time of the daily endogenous peak (i.e. acrophase) of the rhythm of plasma prolactin concentration or a function of the time of daily injection of exogenous hormone (or of a substance that increases prolactin levels) or of the relation between endogenous peak and any induced peak. Furthermore, high levels of prolactin restricted to a discrete daily interval have a much greater physiologic (e.g. metabolic) effect in animals than do constant high levels throughout a day (Cincotta, A. H. et al., Horm. Metab. Res. 21:64-68, 1989; Borer, K. T. in The Hamster: Reproduction and Behavior (ed. Siegel, H. I.) 363-408, 1985). Such findings demonstrate the existence of daily response rhythms to prolactin by certain types of cells.

One early demonstration of a daily variation in physiological responsiveness to any hormone was the dramatic variation in fattening responsiveness to prolactin in the white-throated sparrow (Meier, A. H. et al., Gen. Comp. Endocrinol. 8:110-114, 1967). Injections at midday of a 16-hour daily photoperiod stimulated 3-fold increases in body fat levels, whereas injections given early in the photoperiod reduced fat stores by 50%. Such daily variations in fattening responses to prolactin have subsequently been demonstrated in numerous species of all the major vertebrate classes (Meier, A. H., Amer. Zool. 15:905-916, 1975; Meier, A. H., Hormonal Correlates of Behavior (eds. Eleftherton and Sprott) 469-549, 1975) indicating the fundamental nature of such a temporal organization. The fattening response rhythm persists under constant light conditions (Meier, A. H. et al., Proc. Soc. Exp. Biol. Med. 137:408-415, 1971) indicating that it, like many other endogenous daily variations, is a circadian rhythm.

Additional studies have demonstrated that circadian rhythms have primary roles in regulating numerous physiologic activities, such as immune function, lipid metabolism, and body fat stores (Cincotta, A. H. et al., Endocrinology 136(5):2163-2171, 1995; Meier, A. H. et al., Current Ornithology II (ed Johnston R. E.) 303-343, 1984; Meier, A. H., Amer. Zool. 15:905-916, 1975; Meier, A. H., Hormonal Correlates of Behavior (eds. Eleftherton and Sprott) 469-549, 1975; Meier, A. H. et al., J. Am. Zool. 16:649-659, 1976); Cincotta et al., Life Sciences 45:2247-2254, 1989; Cincotta et al., Ann. Nutr. Metab. 33:305-14, 1989; and Cincotta et al., Horm. Metabol. Res. 21:64-68, 1989.

The immune function studies (Cincotta, A. H. et al., Endocrinology 136(5):2163-2171, 1995) showed that the responsiveness of immune system components to prolactin is time of day dependent. Timed daily administrations of prolactin or bromocriptine were shown to be able to stimulate or inhibit immune responses, depending on the time of day that they are administered. That is, there was found to be a specific window of time during which prolactin was found to have an immunostimulatory effect, outside of which prolactin exerted no effect. Conversely, there was found to be a specific window of time during which bromocriptine, a prolactin inhibitor, was found to have an immunosuppressive effect, outside of which it had no effect on immune function. These findings indicate an essential role for prolactin rhythms in the regulation of immunity.

The experiments relating to metabolism showed that an interaction of circadian rhythms of liporegulatory hormones (stimuli) and of circadian responses to these hormones (in target cells) determines amount of lipogenesis and fat storage. Thus, high plasma concentrations of prolactin (which serves as the stimulus) occur during the daily interval of maximal fattening responsiveness to prolactin in fat animals, but occur at other unresponsive times of day in lean animals (Meier, A. H., Amer. Zool. 15:905-916, 1975; Meier, A. H., Hormonal Correlates of Behavior (eds. Eleftherton and Sprott) 469-549, 1975; Speiler, R. E. et al., Nature 271:469-471, 1978). Similarly, plasma insulin (which acts as the stimulus) levels are highest during the daily interval of greatest hepatic lipogenic response to insulin in obese hamsters, but at a different time of day in lean hamsters (deSouza, C. J. et al., Chronobiol. Int. 4:141-151, 1987; Cincotta, A. H. et al., J. Endocr. 103:141-146, 1984). The phase relationships of these stimulus and response rhythms are believed to be expressions of neural circadian centers which in turn can be reset by neurotransmitter agents and hormone injections (including prolactin) to produce either fat or lean animals (Meier, A. H., Trans. Am. Fish. Soc. 113:422-431, 1984; Meier, A. H. et al., Current Ornithology II (ed Johnston R. E.) 303-343, 1984; Cincotta, A. H. et al., J. Endocrinol. 120:385-391, 1989; Emata, A. C. et al., J. Exp. Zool. 233:29-34, 1985; Cincotta, A. H. et al., Chronobiol. Int'l 10:244-258, 1993; Miller, L. J. et al., J. Interdisc. Cycles Res. 14:85-94, 1983). Accordingly, timed prolactin administration or enhancement has been shown to act directly upon tissues (e.g. liver in lipogenesis) undergoing circadian rhythms of responsiveness to the hormone to produce immediate variations in net physiologic effects (Cincotta, A. H. et al., Horm. Metab. Res. 21:64-68, 1989) and also acts indirectly by resetting one of the circadian neuroendocrine oscillations of a multi-oscillatory circadian pacemaker system to establish different phase relations between the multiple circadian (neural, hormonal, and tissue) expressions that control lipid metabolism (Meier, A. H., Trans. Am. Fish. Soc. 113:422-431, 1984; Meier, A. H. et al., Current Ornithology II (ed Johnston R. E.) 303-343, 1984; Cincotta, A. H. et al., J. Endocrinol. 120:385-391, 1989; Emata, A. C. et al., J. Exp. Zool. 233:29-34, 1985; Cincotta, A. H. et al., Chronobiol. Int'l 10:244-258, 1993; Miller, L. J. et al., J. Interdisc. Cycles Res. 14:85-94, 1983).

It has previously been shown that prolactin, or substances that affect circulating prolactin levels, also affect circadian rhythms and in fact can be used to modify such rhythms (so that they more closely resemble the rhythms of lean, healthy, young individuals of the same sex) and to reset such rhythms (so that the modified rhythms persist in the modified condition). See, e.g. U.S. patent applications Ser. Nos. 08/158,153, 07/995,292, 07/719,745, 07/999,685 08/171,569, and U.S. Pat. No. 5,344,832. This prior work by the present inventors has been clinically tested in humans afflicted with various physiological disorders (obesity, diabetes, atherosclerosis, hypertension, immune dysfunction, and others) with meaningful results.

In particular, in U.S. patent application Ser. No. 07/995,292 (now allowed), and in its continuation-in-part Ser. No. 08/264,558, filed Jun. 23, 1994, the present inventors disclose a method for the reduction in a subject, vertebrate animal or human, of body fat stores, and reduction of at least one of insulin resistance, hyperinsulinemia, and hyperglycemia, and other metabolic diseases, especially those associated with Type II diabetes. More specifically, the foregoing applications disclose methods for: (i) assessing the daily prolactin level cycles of a normal (healthy) human or vertebrate animal (free of obesity, disease or other disorder); (ii) diagnosing aberrant daily prolactin level cycles of a human or vertebrate animal; and (iii) determining the appropriate adjustments that need to be made to normalize such aberrant prolactin level cycles. This method involves the administration of at least one of a prolactin reducer and/or a prolactin enhancer at a first predetermined time (or times) within a 24-hour period (if only a prolactin reducer is administered) and/or at a second predetermined time (or times) of a 24-hour period (if a prolactin enhancer is administered). This therapy, when continued for several days, weeks or months, results in the long-term adjustment of aberrant or abnormal prolactin level cycles so that they conform to (or approach) normal prolactin level cycles. In most cases, this benefit persists over the long-term even after cessation of therapy. As a result, aberrant physiological parameters associated with various metabolic disorders are restored to normal levels or are modified to approach normal levels.

Further illustration of the utility of resetting prolactin rhythms can be found in U.S. patent application Ser. No. 08/271,881, filed Jul. 7, 1994, a method for regulating immune function by resetting prolactin rhythms is disclosed, and in U.S. patent application Ser. No. 08/475,296 filed Jun. 7, 1995, a method for arresting the growth of or eradicating neoplastic growths in mammals having daily prolactin rhythms is disclosed.

Melatonin

Other studies have shown that melatonin levels tend to be altered in humans suffering from tumors (Bartsch, C. et al. Ann. N.Y. Acad. Sci. 719:502-525, 1994) and that sera from tumor-bearing animals can suppress melatonin secretion by pineal organ cultures (Leone, A. M. et al., J. Pineal Res. 17:17-19, 1994). Thus, while other studies have correlated tumors with abnormal melatonin levels and secretion, there is no teaching in the prior art of the desirability of adjusting abnormal melatonin daily rhythms in mammals in order to inhibit or destroy tumors.

### Photodynamic Therapy (PDT)

PDT is a promising new approach for the selective eradication of tumors which does not result in the deleterious side effects that are often experienced with both chemotherapy and ionizing radiation therapy. PDT involves the systemic administration of tumor localizing photosensitizers that can kill malignant tissue when irradiated with light of the appropriate wavelength. Photoactivation of photosensitizers in the presence of oxygen generates highly reactive and cytotoxic molecular species by one or both of the following mechanisms:

(a) a type I reaction where the excited state of the dye interacts directly with biomolecules to generate free radicals, hydrogen peroxide, superoxide, etc.; or

(b) a type II reaction where the excited state of the dye interacts directly with oxygen to generate the highly reactive, short-lived cytotoxin singlet oxygen (¹O₂).

In vitro these oxidizing species cause cell death as a result of damage to various cellular organelles and functions depending on the photosensitizer used. In vivo, however, studies of the acute effects of PDT on animal tumors using a variety of sensitizers have shown that vascular occlusion is largely responsible for tumor eradication. This treatment modality has progressed to phase III clinical trials using two PDT drugs, hematoporphyrin (HPD) and Photofrin II (PI). Although encouraging results have been obtained with these PDT agents for a wide variety of tumors, it has become apparent that in order to realize the full potential of PDT additional sensitizers needed to be developed. The reported limitations of both HPD and PII include:

(a) a low absorption coefficient in the region where activating light penetrates tissue most efficiently (600-900 nm);

(b) both products are not a single entity but consist of mixtures of porphyrin ether and ester oligomers;

(c) prolonged retention in the skin leads to dermal photosensitization that can persist for months; and finally

(d) the rapid formation of hypoxic cells which occurs as a consequence of the vasculature damage during PDT with these photosensitizers increases the probability that a fraction of the tumor cells will escape direct photodestruction. Nutritional resupply to these still viable tumor cells through diffusion or angiogenesis may rapidly repopulate the tumor (Henderson et al., Photochem. Photobiol. 49:299-304, 1989; Henderson et al., Cancer Res. 47:3110-3114, 1987).

The success of PDT and the limitations of HPD and PII have stimulated the search for more efficacious phototoxic compounds primarily within the porphyrin family (i.e. benzoporphyrins, chlorins, purpurins, phthalocyanines, etc.); thus these second generation drugs tend to have similar PDT properties, i.e., there is not a great deal of differentiation in the accumulation of the photosensitizers in normal vs. tumor cells, or in mode of cell killing, (i.e. vascular occlusion as for HPD and Photofrin II). An approach of the present inventors has been to study diverse classes of photosensitizers which possess intrinsically different physicochemical and pharmacological properties and to synthesize a large number of novel photosensitizing chromophores for PDT (described in Foley et al., U.S. Pat. No. 4,962,197).

In vitro investigations have established that the cyanine type, phthalocyanine type, porphyrin type, and benzophenoxazine analog dyes of the present invention possess characteristics which allow for their beneficial use in the treatment of tumors (Richter, A. M. et al., J. Nat. Cancer Inst., 79:1327-1332, 1987; Foultier, M -T., et al. J. Photochem. Photobiol. B:Biol., 10:119-132, 1991; Morgan, A. M., et al. Future Directions and Applications in Photochemistry and Photobiology, SPIE Institute Series, Vol. IS 6:87-106, 1990). These include: (a) a high degree of lipophilicity; (b) rapid tumor localization; (c) absorption of light in a spectral region where light penetrates tissue maximally; and (d) efficient generators of phototoxin. The benzophenoxazine analogs seem to rapidly accumulate intracellularly and cause tumor destruction with minimal damage to the vasculature, unlike HPD or Photofrin II. This is believed to potentiate the effect of the therapy because oxygen supplied to tumor cells is required for the cytotoxic effects of PDT including benzophenoxazine analogs (Cincotta et al., Cancer Res. 54:1249-1258, 1994; Lin, C -W. et al., Cancer Res. 51:1109-1116, 1991; Foster, T. H., et al., Radiation Res. 126:296-303, 1991; Foster, T. H. et al., SPIE Proc. 1645:104-114, 1992).

### SUMMARY OF THE INVENTION

It has long been known that mammals (including humans) suffering from tumors have abnormal prolactin profiles. It has been known for quite some time that melatonin levels are also abnormal in mammals suffering from tumors. As disclosed in copending U.S. patent application Ser. No. 08/475,296 filed Jun. 7, 1995, it has recently been unexpectedly discovered that the growth of tumors in mammals (including humans) may be treated by modifying the abnormal prolactin profile of the mammal afflicted with tumors so that the profile approaches or conforms to the prolactin profile of a lean, young healthy mammal of the same species and sex (the normal profile). It was shown that the abnormal prolactin profile of the afflicted mammal may be modified by:

(i) direct administration of prolactin,

(ii) adjusting the prolactin profile by timed administration of prolactin modulators, i.e. prolactin enhancers and/or reducers, or by

(iii) resetting the circadian rhythm of the afflicted mammal to a normal phase and amplitude through the timed administration of prolactin enhancers and prolactin reducers (such as bromocriptine).

It has also been known that PDT is a promising method of treating tumors without the severe side effects of standard chemotherapy and ionizing radiation.

It has now been surprisingly and unexpectedly discovered that the growth arrest or eradication of tumors that can be achieved with PDT can be significantly augmented by normalizing one or both of the prolactin and melatonin profiles of the tumor bearing mammal to the respective normal profiles for a mammal of the same species and sex. This discovery was entirely unexpected because (a) the mechanism of action of PDT is completely unrelated to and independent of the mechanism for adjustment of prolactin and melatonin daily rhythms, and (b) there has been no previous report of treating tumors in mammals by normalizing melatonin daily rhythms.

The photodynamic therapy of the present invention is used to treat solid neoplastic tumors including by way of non-limiting example sarcomas, carcinomas, and gliomas. Among the specific neoplastic tumors that have been successfully treated (i.e. reduced in size or eliminated entirely) using PDT are papillary bladder tumors, lung cancer, obstructing esophogeal tumors, gastric, colon, and cervical cancers. Metastatic breast cancer tumors can also be treated with the present invention, as well as squamous cell and basal cell skin cancers. The tumor to be treated according to the present invention must be accessible to a source of actinic light. Thus the invention can be practiced on surface lesions. Tumors in internal organs may be treated using, for example, fiber optic devices to enable exposure of the tumors to actinic radiation. The tumors referred to in this specification are all malignant tumors.

Thus, one aspect of the present invention is a method for inhibiting or eliminating tumors in mammals by administration to the mammal of a prolactin reducer and/or enhancer or a timed sequential administration of a prolactin reducer and enhancer at a predetermined time or times during a 24-hour period that results in modification of the mammal's abnormal prolactin profile so that it approaches or conforms to the prolactin profile of a young healthy mammal of the same species and sex, said administration occurring before or during treatment with PDT.

Another aspect of the present invention is directed to a method for treating or inhibiting tumors in mammals on a long-term basis by continuing the foregoing timed administration(s) of prolactin reducer and/or enhancer after tumor reduction or eradication by combined neuroendocrine adjustment/PDT treatment until the altered prolactin rhythm of the subject is reset to a normal rhythm and persists in this reset condition for an extended period of time even after cessation of therapy, resulting in persistence of either inhibition of tumor growth or persistence of a tumor free state.

A further aspect of the present invention is directed to a method for inhibiting or eliminating tumors in mammals by administration to the mammal of melatonin at a predetermined time or times during a 24-hour period that results in modification of the mammal's abnormal melatonin profile so that it approaches or conforms to the melatonin profile of a young healthy mammal of the same species and sex, said administration occurring before or during treatment with PDT.

Another aspect of the present invention is a method for inhibiting or eliminating tumors in mammals by administration to the mammal of a prolactin modulator and melatonin at a predetermined time or times during a 24-hour period that results in modification of the mammal's abnormal prolactin and melatonin profiles so that they approach or conform to the corresponding prolactin and melatonin profiles of a young healthy mammal of the same species and sex, said administration occurring before or during treatment with PDT.

Thus, the present invention is directed to treating or inhibiting the growth of tumors in mammals by adjusting the circadian rhythms of prolactin and melatonin, and administering PDT.

Advantages of the present invention include:

enhanced reduction in tumor growth and accelerated eradication of tumors.

the ability to inhibit or eradicate malignant tumors without the debilitative effects of chemotherapeutic agents or ionizing radiation.

the tumor growth inhibiting and treatment benefits of the present invention may persist long-term even after the administration of prolactin, melatonin, and PDT have been discontinued.

Other features and advantages of the present invention will be apparent from the following description taken in conjunction with the accompanying drawings.

### BRIEF DESCRIPTION OF THE DRAWINGS

FIG. 1 depicts the normal or baseline prolactin profile for healthy male and female humans.

FIG. 2 is the prolactin daily rhythm or profile curve for breast cancer patients with tumors.

FIG. 3 is the normal melatonin daily rhythm or baseline melatonin profile for healthy male humans.

FIG. 4 is the melatonin daily rhythm or profile curve for men with prostate cancer.

FIG. 5 is a bar graph illustrating the effects of prolactin adjustment therapy alone, PDT alone, and the combination of prolactin adjustment and photodynamic therapies on tumor size in the EMT-6 implanted tumor mouse model system.

FIG. 6 shows representative porphyrin photosensitizers (BPD-MA, Mono-L-Aspartyl Chlorin e6, Tin etiopurpurin) and phthalocyanine photosensitizers (aluminum, zinc, and silicon pthalocyanine).

FIG. 7 shows representative benzophenoxazine analog photosensitizers (EtNBS, Dye 4-115), sulfated phthalocyanine, and cyanine photosensitizers (EDKC, pyrilium dyes, merocyanine 540).

### DETAILED DESCRIPTION OF THE INVENTION

All patents, patent applications, and literature references discussed in this specification are hereby incorporated by reference. In case of a conflict in terminology, the present disclosure including its definitions controls.

As used in this specification, the following terms are intended to have the meanings set forth below.

Benzophenoxazine analogs include benzophenoxazines, benzophenothiazines, and benzophenoselenazines.

The terms “dye”, “photosensitizer”, and “chromophore” are used interchangeably.

“Prolactin reducer” refers to a substance or composition that has the ability to lower circulating prolactin levels upon administration to a mammal; “Prolactin enhancer” refers to a substance or composition that has the ability to raise circulating prolactin levels upon administration to a mammal, and includes prolactin itself.

Prolactin reducers and prolactin enhancers are referred to collectively as “prolactin modulators”.

“Prolactin profile” of a subject is a depiction of circulating prolactin levels and their variation over all or part of a 24 hour period, and therefore is expression of all or part of the subject's plasma prolactin daily rhythm.

“Melatonin profile” of a subject is a depiction of circulating melatonin levels and their variation over all or part of a 24 hour period, and therefore is expression of all or part of the subject's plasma melatonin daily rhythm.

“Hormonal rhythms” include, but are not limited to, the variation over all or part of a 24 hour period of blood levels of prolactin and melatonin.

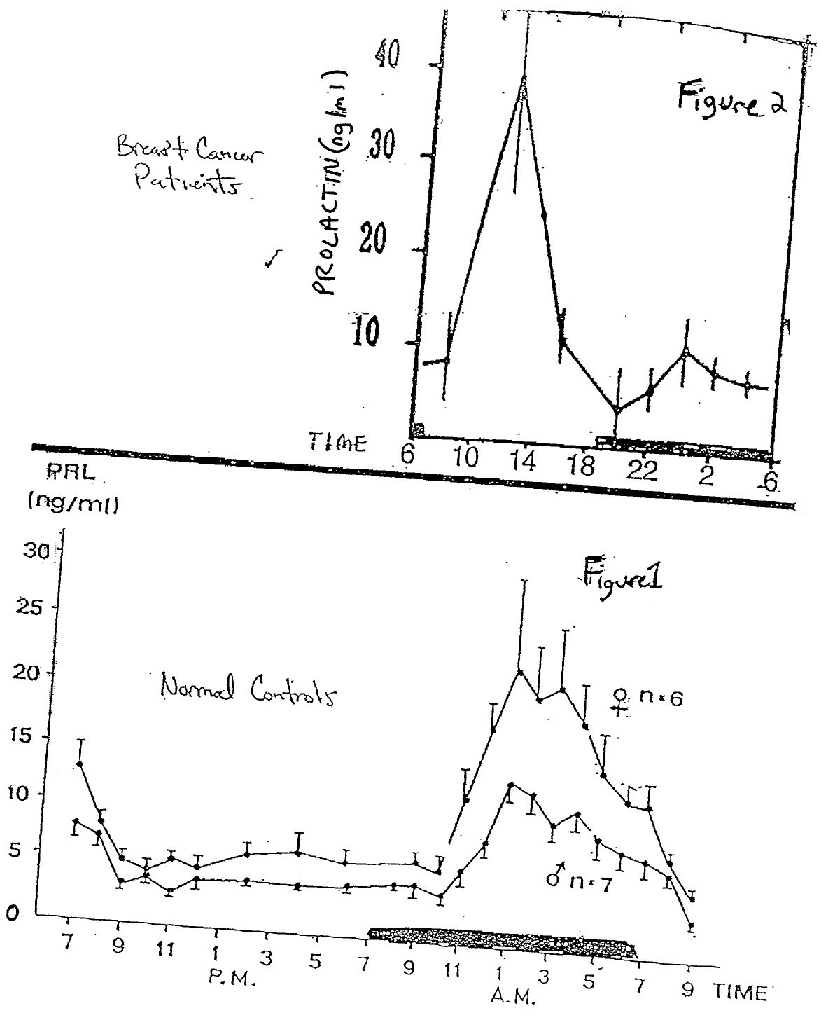

The term “healthy” refers to a young, lean subject free of disease including malignancies, tumors, immune system dysfunctions and metabolic abnormalities. A healthy subject is one with normal prolactin and melatonin profiles, i.e., profiles that does not depart from the baseline of that subject's species and sex by more than one standard error of the mean (SEM). The normal or baseline prolactin profile for healthy male and female humans is depicted in FIG. 1. The normal or baseline melatonin profile for healthy male humans is depicted in FIG. 3.

### Determination and Adjustment of Prolactin Rhythms

In order to avoid “false positives” a subject will not generally be considered to have an abnormal prolactin profile unless:

(a) the subject's daytime blood prolactin level is at least 1 standard error of the mean (SEM) higher than the baseline at two (or more) time points during daytime spaced apart by at least one and preferably by at least two hours; or

(b) the subject's daytime blood prolactin level is at least 2 SEM higher than the baseline at one time point during daytime; or

(c) the subject's night time blood prolactin level is at least 1 SEM below the base line at two (or more) time points spaced apart (as in (a)); or

(d) the subject's night time blood prolactin level is at least 2 SEM below the base line at one time point during night time.

The human male and female prolactin baselines are depicted in FIG. 1. One SEM during waking hours (07:00-22:00) is about 1-2 ng/ml for males and about 1-3 ng/ml for females; one SEM during night time (22:00-07:00) is about 3 ng/ml for males and about 3-6 ng/ml for females.

The characteristics of the prolactin level daily rhythm or profile that are to be approached or conformed to in humans include achieving low prolactin levels (2-7 ng/ml of plasma) for males and 2-10 ng/ml for females) during most or all of the time period between 07:00 and 22:00 h.

Ideally, a peak prolactin level in humans should also be achieved between the hours of 22:00 and 07:00 (preferably between 1:00 and 4:00) (the peak should be at least 10 ng/ml and most preferably between 10-15 ng/ml for males and at least 15 ng/ml and preferably between 15 and 25 ng/ml for females).

Effects of Prolactin Modulators on Solid Tumors

The present invention provides an improved method for treating and inhibiting the growth of tumors in mammals having a tumor burden and a prolactin daily rhythm.

The method involves:

(a) adjusting the prolactin profile of the mammal to conform to or approach the normal prolactin profile for healthy members of the same species and sex as said mammal; and then

(b) contacting the cells of said tumor with a photosensitizer and exposing the contacted tumor cells to light of a predetermined wavelength, power density, and energy level.

Thus, one aspect of this treatment is the administration of a prolactin modulator(s) to the tumor bearing mammal at a predetermined time(s) during a 24-hour period. The time for administration of the prolactin modulator is selected so as to adjust the prolactin profile of the mammal receiving treatment to conform or approach the prolactin profile of a healthy mammal of the same sex and species.

It has been found that administration of prolactin enhancers is inhibitory to tumor growth in mammals when given at timed intervals during a 24 hour period which correspond to the peak of prolactin secretion in healthy mammals. Timed prolactin injections in tumor bearing mice which have had their circadian rhythms synchronized with a defined photoperiod were shown to exhibit a decreased tumor burden as compared with tumor bearing mice which did not receive timed prolactin injections. It has also been found that the effect of in vivo prolactin modulation on in vivo tumor growth is time-of-day dependent.

A time-of-day dependent role for prolactin in inhibiting tumors is found when experiments are performed on mice which decrease prolactin blood levels (by administration of a prolactin reducer) during specific daily intervals of lack of tumor growth inhibitory response to exogenous prolactin. Administration of bromocriptine, a D2 dopamine agonist which inhibits endogenous prolactin secretion, increases the inhibition of tumor growth when it is administered at times during a 24 hour period predetermined to reduce prolactin levels to those found in healthy animals of the same sex and species during such time period. The use of bromocriptine for inhibiting tumor growth is shown in Example 4.

The use of melatonin for inhibiting tumor growth is shown in Example 5 . In this example the melatonin blood levels of mice are increased by the administration of melatonin, at a predetermined time known to be the interval of increased responsiveness to melatonin. It is found that the administration of melatonin at the time during a 24 hour period when melatonin levels are peaking in healthy mice exerts a potent inhibitory effect on growth of tumors.

These examples establish the ability of prolactin and melatonin to modulate tumor growth, and the relationship between tumor growth inhibition, endogenous prolactin levels (or prolactin enhancers or reducers), and the time of day of prolactin reduction or enhancement.

Although the foregoing experiments are conducted in mice, they are dependent on features of physiology that are common to mammals having a prolactin daily rhythm including humans. These experiments demonstrate that blood levels of prolactin and melatonin can be manipulated during predetermined intervals to bring about a desirable result with regard to inhibition of growth of tumors.

According to the method of the present invention, the alteration of prolactin levels of a subject at particular times of day provides methods for inhibiting tumor growth in the subject or inhibiting the growth of metastases in a subject. The method may be used on all types of tumors, including but not limited to sarcomas, carcinomas, glioblastomas, melanomas, basal and squamous cell carcinomas, lymphomas, adenomas, and leukemias.

It is known that young adult healthy mammals of a given species (and sex), e.g. humans (suffering from no hormonal or metabolic disorders or cancer or other infection or ailment) have highly predictable daily prolactin level rhythms or profiles. The baseline curve for healthy human males and females in FIG. 1 is derived from such young healthy individuals.

The phase relationship between the daily peaks of the stimulus (plasma prolactin) rhythm and response (tumor growth inhibition) to prolactin has been found to be important in tumor growth inhibitory activity. Environmental and pharmaceutical factors influencing either of these rhythms can be expected to impact tumor growth.

Humans with solid tumors, such as found in breast cancer and prostate cancer, have perturbed prolactin rhythms, which is apparent in a comparison of the prolactin rhythms of healthy women with the rhythms of women with breast cancer, which rhythms are shown in FIGS. 1 and 2, respectively. Humans with tumors thus may be able to benefit to a significant extent by adjustment of their prolactin daily rhythms (as expressed by their prolactin profile) to conform to or approach the normal or baseline prolactin curve of FIG. 1. An adjusted prolactin profile approaches a normal or healthy profile, if all or a portion of the abnormal profile moves in the correct direction by at least 2 ng/ml.

One approach to adjusting prolactin profiles in a subject is as follows:

(i) the prolactin levels of the tumor bearing human should be ascertained by assaying blood samples of the tumor bearing human at certain spaced apart intervals within a 24 hour period (or portions thereof), and

(ii) the resultant prolactin profile of the tumor bearing human should be compared to the prolactin profile for a healthy human of the same sex. Depending on the difference between (i) and (ii), the adjustment then involves administering one or both of the following:

(a) a prolactin reducer at a first predetermined time (or at more than one first predetermined time) and in a first amount effective to reduce day time prolactin levels if these levels are too high; and

(b) a prolactin enhancer at a second predetermined time (or at a plurality of second predetermined times) and in a second amount effective to increase night time prolactin levels if these levels are too low.

In general, if a prolactin level altering substance is to be administered, appropriate allowance should be made with respect to the time of administration to permit that substance (depending on its pharmacokinetic properties) to affect prolactin levels such that prolactin levels would be modified during the appropriate time of day. Thus, the prolactin altering substance will be administered as follows:

(a) if prolactin is administered, it will be administered, preferably by injection, during the time interval that prolactin levels need to be raised;

(b) if a prolactin enhancer other than prolactin is administered, it will be administered during or some time shortly prior to the time interval when prolactin levels need to be raised (how much prior depends on pharmacokinetic properties: 0-3 hours prior has generally been found to be effective); and

(c) if a prolactin reducer is administered it will also be administered during or slightly prior to the time that prolactin levels need to be reduced (again, 0-3 hours prior has generally been found to be effective).

In the method of the present invention, “prolactin enhancer” includes prolactin as well as substances which increase circulating prolactin levels (e.g. by stimulating prolactin secretion). Non-limiting examples of a prolactin enhancer include prolactin; melatonin; dopamine antagonists such as haloperidol, pimozide, phenothiazine, domperidone, sulpiride and chlorpromazine; serotonin agonists, i.e., MAO-A inhibitors, e.g., synthetic morphine analogs, e.g., methadone; antiemetics, e.g., metoclopramide; estrogens; and various other serotonin agonists, e.g., tryptophan, 5-hydroxytryptophan (5-HTP), fluoxetine, and dexfenfluramine. Moreover, the non-toxic salts of the foregoing prolactin enhancing compounds formed from pharmaceutically acceptable acids are also useful in the practice of this invention. Melatonin and 5-HTP have been found particularly useful in the practice of this invention. Melatonin is particularly useful, because its administration at the appropriate time will also normalize abnormal melatonin rhythms, as shown below.

Nonlimiting examples of prolactin reducers include prolactin-inhibiting dopamine agonists (D2 agonists) such as dopamine and certain ergot-related prolactin-inhibiting compounds. Nonlimiting examples of dopamine agonists are 2-bromo-alpha-ergocriptine;6-methyl-8-beta-carbobenzyloxy-aminoethyl-10-alpha-ergolin;8-acylaminoergolines, are 6-methyl-8-alpha-(N-acyl)amino-9-ergoline and 6-methyl-8 alpha-(N-phenylacetyl)amino-9-ergoline; ergocornine; 9,10-dihydroergocornine; and D-2-halo-6-alkyl-8-substituted ergolines, e.g., D-2-bromo-6-methyl-8-cyanomethylergoline; carbi-dopa and L-dopa; and lisuride. Moreover, the non-toxic salts of the prolactin-reducer compounds formed with pharmaceutically acceptable acids are also useful in the practice of this invention. Bromocriptine, or 2-bromo-alpha-ergocryptine, has been found particularly useful in the practice of this invention.

The modulation of tumor growth inhibition induced by prolactin enhancers or reducers is expected to be dose-dependent over a range of dosages.

In treating mammals, generally, dosages of the prolactin reducer and/or enhancer, respectively, are each given, generally once a day, generally over a period ranging from about one month to about one year, but treatment can continue indefinitely (if necessary or desired) for months or even several years. The preferred prolactin reducer (accelerated release bromocriptine) is given at daily dosage levels ranging from about 3 micrograms to about 300 micrograms, preferably from about 10 micrograms to about 100 micrograms, per kg. of body weight, and a preferred prolactin enhancer, melatonin, is given at daily dosage levels ranging from about 10 micrograms to about 800 micrograms, preferably from about 10 micrograms to about 200 micrograms, per kg. of body weight per day to modify, or alter, the prolactin profile. Another preferred prolactin enhancer, 5-hydroxytryptophan, is given at daily dosage levels ranging from about 500 micrograms to about 13 milligrams per kg. of body weight, preferably from about 500 micrograms to about 2.5 milligrams per kg. of body weight. The exact dosage within these ranges to be administered to each subject will depend upon the particular prolactin modulator, the subject's age, stage of disease, physical condition and responsiveness to treatment.

In order to adjust the prolactin profile of a mammal, administration of either or both prolactin altering substances can be continued for a time sufficient to reset the circadian plasma prolactin rhythm to the phase and amplitude to that of a healthy subject of the same sex and species at which time treatment may be discontinued. If the subject suffers a relapse, treatment may be resumed in order to adjust the prolactin profile of the subject to conform or approach the prolactin profile of a healthy subject of the same sex and species. The time needed for resetting varies but is generally within the range of one month to one year. For some patients (e.g. patients in particularly poor physical condition, or those of an advanced age) it may not be possible to reset their prolactin rhythm within the above time periods and such patients may require a longer, or even continuous, treatment with prolactin enhancers and/or reducers. The dosage and timing information set forth above is designed for bromocriptine, melatonin, and 5-hydroxytryptophan and will have to be altered for other agents using the dosage and timing methodology disclosed herein.

In the practice of this invention, a prolactin reducing compound, and/or a prolactin enhancer are administered daily to a subject preferably orally, or by subcutaneous, intravenous or intramuscular injection. The reducer or enhancer can also be administered by inhalation. Dermal delivery systems e.g., skin patches, as well as suppositories and other well-known systems for administration of pharmaceutical agents can also be employed. Treatment generally lasts between about one month and about one year on average in humans. The administration of the prolactin reducer and/or prolactin enhancer in this manner will thus reset the phase and amplitude of the neural oscillators that control the body's ability to inhibit tumor growth to facilitate inhibition of tumor growth on a long term basis (e.g., several months or years). An improvement in the ability to inhibit tumor growth can be assessed by observation of partial or total ablation of the tumor or metastatic regrowth after the removal of a primary tumor. Instead of measuring tumor burden directly, well-known assays of tumor burden (e.g. assays of tumor-specific antigens, magnetic resonance imaging, CAT scanning, X-rays, ultrasound, counting blood-borne tumor cells in blood samples, etc.) can be used to assess the effect of treatment with timed administration of prolactin modulators.

Another approach to adjusting a cancer patient's abnormal profile is to follow these more specific guidelines to initially determine prolactin modulator administration timing, for a period of treatment of approximately 26 weeks for human subjects:

(i) Give prolactin reducers from 0600 hours to 1000 hours in a dosage range sufficient to decrease diurnal prolactin levels to within 1 SEM of the normal range of diurnal prolactin levels found in humans without tumors.

(ii) Give prolactin enhancers before or at bedtime in a dosage range sufficient to increase serum prolactin levels to at least the level of a normal, healthy human without tumors.

The aspect of the invention directed to an inhibition of tumor growth by resetting the prolactin profile of a mammalian subject (animal or human) having an aberrant prolactin profile to conform to or approach the prolactin profiles for young healthy members of the same species and sex (e.g. the baselines of FIG. 1) involves administration of a prolactin reducer, or a prolactin enhancer, or both, at predetermined dosages and times dictated by the aberrant (pre-treatment) prolactin profile of the subject to be treated. The amounts of prolactin reducers and/or enhancers that are required to bring about this modification are within the same ranges as set forth above, but the time(s) of administration of these prolactin modulator(s) is determined by reference to how much and when the aberrant profile differs from the normal prolactin profile (baseline curve). Methods for determining the amounts and timing of administration are also set forth in copending U.S. patent application Ser. No. 07/995,292 (now allowed) and its C-I-P, Ser. No. 08/264,558 filed Jun. 23, 1994, both incorporated by reference.

Another approach to normalizing the prolactin rhythm of a cancer patient by adjusting an abnormal prolactin profile is to give up to 4.8 mg/day of bromocriptine as follows; 0.8 mg/day for each of the first 7 days; beginning on day 8 and for 7 days thereafter, 1.6 mg/day is administered to the patient; beginning on day 15 and for 7 days thereafter, 2.4 mg/day are administered; beginning on day 22 and for 7 days thereafter, 3.2 mg/day is administered; beginning on day 29 and for 7 days thereafter, 4.0 mg/day is administered and beginning on day 36 and for 7 days thereafter, 4.8 mg per day is administered for 7 consecutive days. A preferred accelerated release bromocriptine dosage form has been disclosed in copending U.S. patent application Ser. No. 08/171,897 also incorporated by reference.

### Determination and Adjustments of Melatonin Daily Rhythms

Healthy (normal) subjects, i.e., lean members of a species not suffering from tumors or any other pathologies have highly predictable daily melatonin profiles, which in humans have a characteristic sharp rise to a peak in the hours following the onset of sleep (23:00 to 4:00). “Healthy” individuals have melatonin profiles that are at within 1 SEM of the normal melatonin profile of FIG. 3, preferably for at least four melatonin levels measured at different times or within 2 SEM of the normal melatonin profile for at least two measured melatonin levels.

Normal daily melatonin profiles can be determined by methods identical to those described for prolactin profiles, except that blood samples are assayed for melatonin rather than prolactin. The daily melatonin profile of a tumor-bearing subject can also be determined by the methods described for prolactin, assaying blood samples for melatonin instead of prolactin.

Once a diurnal melatonin level profile has been developed for an individual, the profile is compared to the “normal” profile (e.g., the one generated as described in the previous section or to FIG. 3). A determination can then be made based on the following general criteria: from about 23:00 h till about 04:00 h, i.e., during the sleeptime peak of the normal daily melatonin profile, the individual's melatonin profile must first have a peak at about the same time or within two to six hours after sleep initiation as the “normal” melatonin peak for subjects in the same category (usually about 02:00-03:00) and must also be within one SEM of the normal healthy melatonin profile (preferably for four melatonin readings or alternatively within two SEM for at least two melatonin readings).

To determine if a subject has an aberrant melatonin profile, the bedtime on the subject's melatonin profile should ideally be coincident with the bedtime on the profile of normal subjects. If this is not the case, the profile of the subject and the profile of normal individuals can be superimposed and one or the other can be shifted so that the sleep initiation time of the subject to be tested coincides with the sleep initiation time of normal healthy subjects.

Determination of Treatment for an Affected Subject

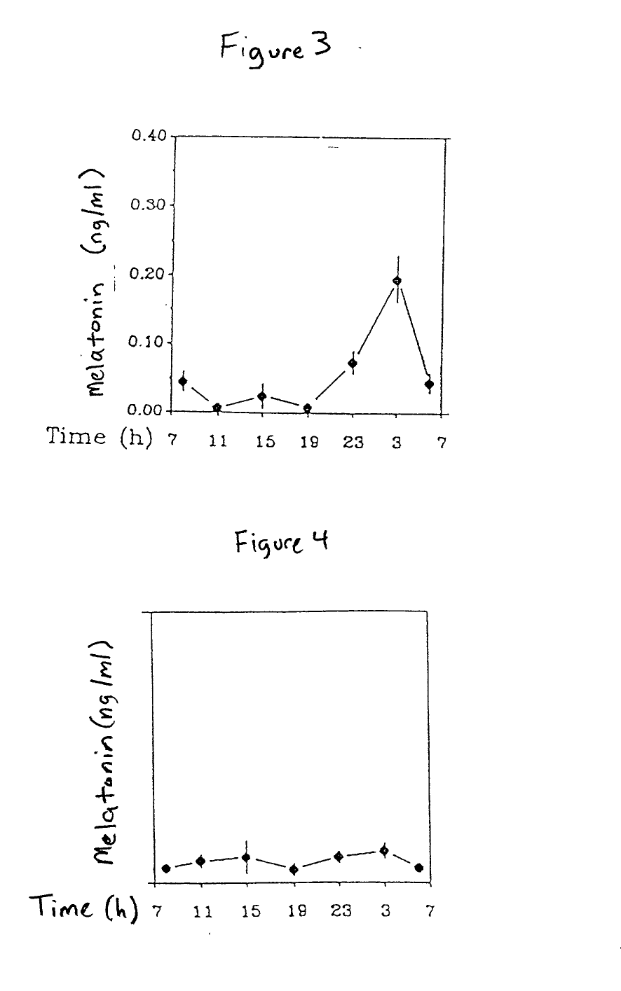

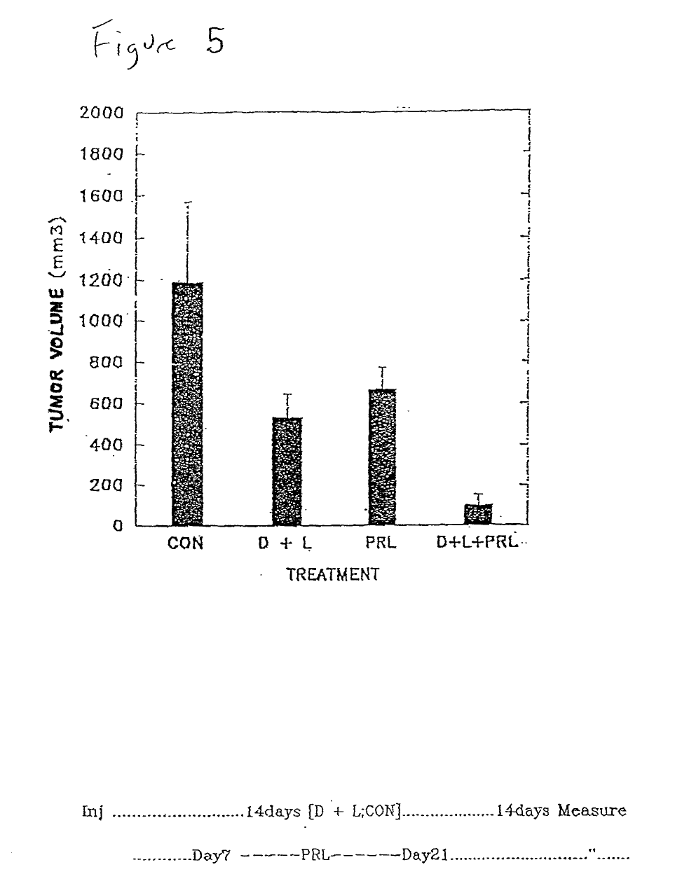

The information (melatonin profile or set of sleeptime melatonin levels) generated as described above is used to determine the type and extent of adjustment required. In general, those individuals that have tumors display abnormal melatonin profiles (or sleeptime melatonin levels) as compared to healthy individuals (compare, e.g., FIGS. 3 and 4). By adjusting the abnormal melatonin profile of such individuals by administration of melatonin or a melatonin enhancer at the appropriate time of day and in the appropriate dosage (amount) it is possible to adjust such individuals' melatonin profile to conform (or at least approach) a normal profile. The amount and timing of administration of such dosages can be determined based upon information contained in the melatonin profiles (or sleeptime melatonin levels) discussed above, and based on the time it takes for the administered melatonin or melatonin enhancer to raise the melatonin levels in the subject's bloodstream.

An adjusted melatonin profile approaches a normal or healthy profile if all or a portion of the abnormal profile moves in the correct direction by at least 0.1 pmol/ml. For example, if a human subject's abnormal melatonin level is 0.01 pmol/ml between 24:00 and 01:00 and (after adjustment) it is increased to 0.11 pmol/ml during the same time period, the adjusted profile approaches the healthy profile. It is thus important to increase the area under the sleeptime melatonin curve (by at least about 30% and typically at least about 50%). It is also desirable not to exceed the normal sleeptime melatonin levels by more than 2 and preferably not more than 1 SEM (4 ng/ml and 2 ng/ml of plasma, respectively).

The treatment determination has two aspects: (a) timing of (each) dose of administration; and (b) amount of (each) dose to be administered.

Whether a full 24-hour or full night-time melatonin profile is generated for a subject to be treated, or only key sleeptime melatonin levels are measured, the following more specific guidelines will generally be followed to initially determine melatonin administration timing, for a period of treatment of approximately 10 days to 26 weeks.

Melatonin is administered once a day, at about bedtime. Generally, the daily dosage range by oral administration is from about 10 μg/kg to about 400 μg/kg of body weight; the preferred oral daily dosage is about 10 μg/kg to about 200 μg/kg of body weight. The preferred range is between about 40 μg/kg and 80 μg/kg of body weight. Melatonin is widely available commercially. The foregoing are applicable for setting initial therapy regimens.

The efficacy of a particular regimen on a particular patient and the adjustments (in dosage and timing) required, if any, can be determined by comparing the patient's reevaluation melatonin profile or reevaluation sleeptime melatonin levels with the normal profile (or the “healthy” sleeptime profile levels).

Adjustments to the amount(s) of melatonin administered and possibly to the time of administration may be made as described above based on reevaluations.

The present timed daily treatment is typically continued over a period of time ranging from about 10 days to usually about 180 days, resulting in modification and resetting of the melatonin daily rhythm of the patient to that of a healthy person, at which time treatment may be discontinued. For some patients (e.g. patients in particularly poor physical condition, or those of an advanced age) it may not be possible to reset their melatonin rhythm within the above time periods and such patients may require a longer, or even continuous, treatment with melatonin.

As indicated above, in the practice of the present invention normalization of the melatonin daily rhythm can be and preferably is practiced in conjunction with normalization of the prolactin daily rhythm.

### Use of Photodynamic Therapy in the Present Invention

Preparation of the Photosensitizers

The preferred benzophenoxazine analogs for use in the present invention and the synthesis of the benzophenoxazine analogs are those described in Foley et al., U.S. Pat. No. 4,962,197, which is herein incorporated by reference. The photosensitizer can be purified by medium pressure (100 psi) liquid chromatography using silica gel (Woelm 32-63) as a solid phase and eluting with a linear gradient of methylene chloride:methanol (100:0-90:10). The resulting purified photosensitizer is homogeneous by thin layer chromatography and high field nuclear magnetic resonance spectroscopy (JEOL 400 MHz). Aqueous solutions of the compound can be prepared in isotonic sucrose at a concentration of 0.175 mg/ml.

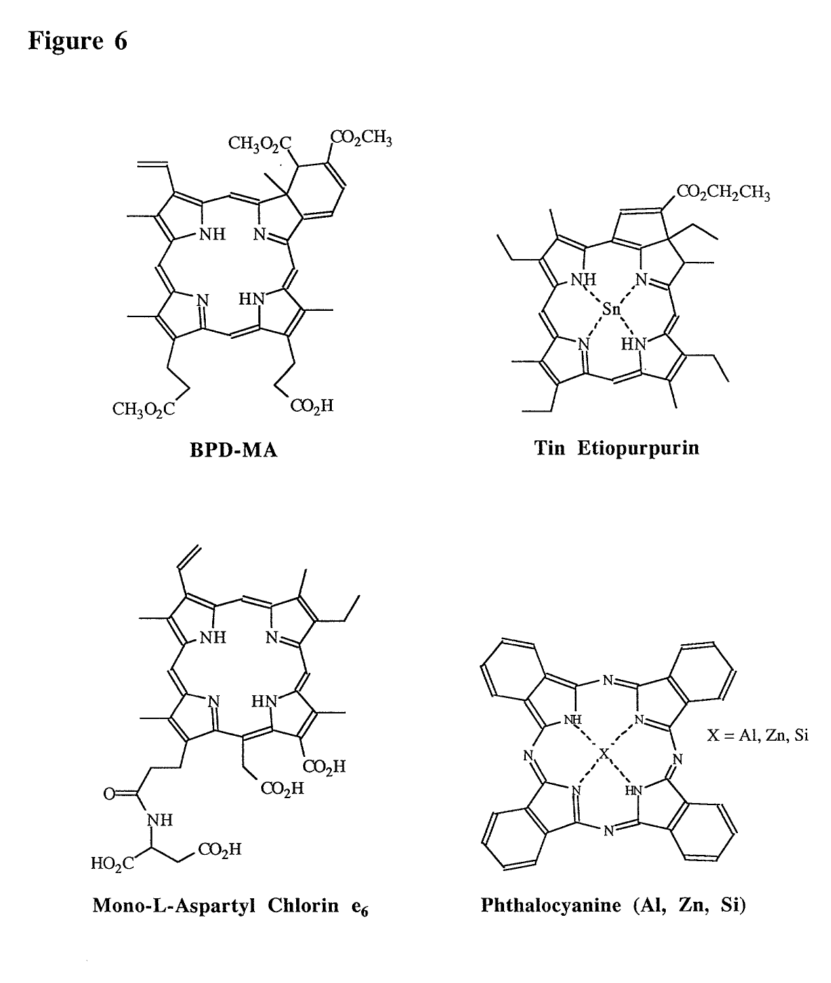

Benzoporphyrin derivative, mono acid ring a (BPD-MA) (FIG. 6) can be made by methods described in Levy et al., U.S. Pat. No. 4,920,143 and Pangka, V. S. et al., J. Org. Chem. 51:1094-1100, (1986), both of which are herein incorporated by reference. BPD-MA can also be obtained from Quadra Logic Technologies, Vancouver, BC, Canada. Methods for the synthesis of representative porphyrin photosensitizers to be used in the present invention can be found in Bommer, J. C. et al., European Patent Application No. 169,831; Shiau, F -Y. Et al., SPIE Institute Series IS6:71-86, 1990; Bonnet, R. Chemical Society Reviews 24:19-33, 1995; and Morgan, A. R. et al., Cancer Res. 48:194-198, 1988), herein incorporated by reference. Many of the porphyrin photosensitizers of the present invention are also commercially available. Monoaspartyl chlorin e6 (FIG. 6) can be obtained from Nippon Petrochemical, Tokyo, Japan. Purpurins, such as tin etiopurpurin (FIG. 6), can be obtained from PDT, Inc., Santa Barbara, Calif. Bacteriochlorins, such as m-tetrahydrophenylchlorin (m-THPC), can be obtained from Scotia Pharmaceuticals, Guildford, England.

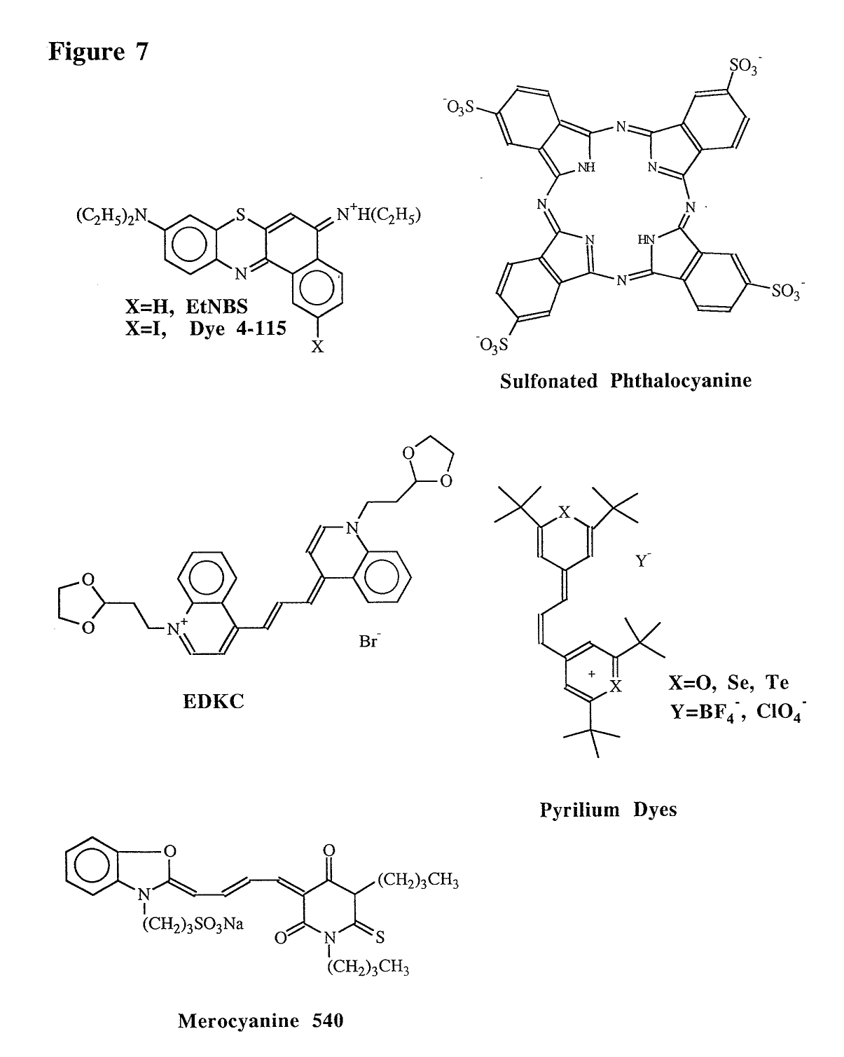

Methods for the synthesis of representative phthalocyanine photosensitizers (FIG. 6 and 7) to be used in the present invention can be found in Oleinick, N. L., et al., Photochem. Photobiol. 57:242-247, 1993; and Bonnet, R. Chemical Society Reviews 24:19-33, 1995, herein incorporated by reference. These compounds are also commercially available from Ciba Geigy, Basel, Switzerland, and Quadra Logic Technologies, Vancouver, BC, Canada.

N,N′-bis(2-ethyl-1,3-dioxolane)kryptocyanine(EDKC) (FIG. 7) can be prepared according to the method of Hamer in The Cyanine Dyes and Related Compounds (John Wiley & Sons, N.Y., 1964) and according to the method described in Oseroff et al., U.S. Pat. No. 4,651,739. EDKC is also commercially available from Molecular Probes Inc., Eugene, Oreg. Merocyanines, such as Merocyanine 540 (FIG. 7) can be prepared according to methods described in Gunther, W. H. H., et al., Phosphorous. Sulfur, and Silicon 67:417-424, 1992; and methods for the synthesis of pyrilium dyes (FIG. 7) can be found in Detty, M. R., et al. Oncology Research 4:367-373, 1993.

The specific photosensitizers listed above are exemplary of the classes of benzophenoxazine analog, porphyrin, cyanine, and phthalocyanine dyes, and are not meant to limit the invention in any way to their sole use.

Photosensitizer Dosage Forms and Administration

The benzophenoxazine analog is preferably a benzophenothiazine or pharmaceutically acceptable salt thereof. Most preferably, the photosensitizer is 5-ethylamino-9-diethylamino-2-iodobenzophenothiazinium chloride (Dye 4-115) (FIG. 7). The benzophenoxazine analog photosensitizer is typically dissolved in sterile isotonic sucrose or saline at 0.1 to 1.0 mg/ml, and preferably 0.25 mg/ml. Administration can be via an intravenous or subcutaneous route.

Administration of benzophenoxazine analog photosensitizer is generally such that between about 0.05 and about 10 mg/kg of body weight of photosensitizer is delivered to the patient, preferably between about 0.1 and about 5 mg/kg of body weight, and most preferably between about 0.5 and about 5 mg/kg of body weight. The active agent is preferably administered by infusion at between 0.1 and 0.5 ml per minute. With all the photosensitizers of the invention, it is preferred that a time interval passes between administration of photosensitizers and photoirradiation (i.e. exposure) of the tumor to light in order to give the photosensitizers time to reach the target tissues and to preferentially dissipate from normal cells, enhancing the differential photosensitizer concentration in tumor cells compared to normal cells. This time interval varies depending on the photosensitizer administered and the route of administration. When a benzophenoxazine analog such as Dye 4-115 is administered intravenously, the time interval is from between 0.5 and 5 hours, and preferably about 1 hour. When a benzophenoxazine analog such as Dye 4-115 is administered subcutaneously, the time interval is between about 0.5 and 5 hours, and preferably about 3 hours.

Administration of other photosensitizers such as porphyrins, phthalocyanines, cyanines, and other benzophenoxazine analogs is carried out using techniques well-known to those of ordinary skill in the art for such chromophores.

Light Activation of Administered Photosensitizers

Light-induced killing of solid tumors according to the invention can be carried out on any solid tumors which are accessible to light from conventional sources (e.g. a xenon arc lamp, an incandescent white light, a projector light source) or from a laser. If a tumor is on the body surface any light source including laser sources can be employed that provides light at the appropriate wavelengths to activate the dyes and that can deliver 50 to 200 mW per square centimeter of treated area. It is preferred to use a tunable argon-dye laser (a 5 watt argon ion pumped tunable dye laser, for example a Coherent, model Innova 100, Palo Alto, Calif.) using DCM (Exiton Chemical Co., Dayton, Ohio). Similar lasers are also commercially available from, for example, Spectra Physics, Mountain View, Calif. However, a projector light source may also be employed. For tumors within the body, which are inaccessible to direct light sources, light is administered via optical fibers and the light source is a laser.

The light to which the tumor is exposed can be broadband white light containing wavelengths of between 600 and 900 nm. The light source must include light at the particular wavelength at which a given photosensitizer generates the most cytotoxin, e.g. singlet oxygen. Using filters, the broadband light can be narrowed to the specific wavelengths which excite particular photosensitizers. When a laser is used, it is tuned to the particular wavelength which most effectively excites a particular photosensitizer, generally the absorption maximum for a particular dye.

The absorption maxima of any particular dye can be determined by methods well-known in the art. Determination of absorption maxima is usually accomplished using a spectrophotometer.

Benzophenoxazine analogs have absorbance maxima at about 630-670 nm. BPD-MA has an absorbance maximum at about 690 nm. Mono-L-aspartyl chlorin e₆ has an absorption maximum at about 664 nm. Tin ethyl etiopurpurin has an absorbance maximum of about 666 nm. m-THPC has an absorbance maximum of about 652 nm. The phthalocyanines have absorption maxima at about 680 nm. EDKC has an absorption maxima at about 700 nm. Pyrilium dyes absorb maximally in about the 450-500 nm range. Merocyanine dyes have absorbance maxima from about 540 to about 626 nm.

In the practice of the invention, when using any of the photosensitizers of the invention, the total light energy delivered when irradiating tumors is between about 5 and about 400 Joules/cm², preferably about 100 Joules/cm². The power density of the light is preferably between about 50 and about 200 mWatts/cm², and is most preferably about 50 mWatts/cm². Delivery of laser light is carried out according to the well-known methods currently used for HPD-mediated laser therapy (Foultier et al., J. Photochem. Photobiol. B. Biol. 10:119-132, 1991). The output beam from the dye laser can be coupled to a quartz fiberoptic cable fitted with a microlens to ensure an even light distribution throughout the treatment field.

### Combined Photodynamic and Neuroendocrine Adjustment Treatment of Tumors

The determination of the presence in a tumor bearing mammal of abnormal prolactin and melatonin rhythms and the adjustment of one or more of the abnormal rhythms to conform to or approach those of a healthy member of the same species and sex is undertaken as described above, comprising administering prolactin reducers or enhancers, and/or melatonin enhancers, singly or in combination, at predetermined time intervals. During or after the hormonal rhythm adjustment treatment, photodynamic therapy is administered as described above. Preferably, photodynamic therapy is administered during the hormonal rhythm adjustment treatment. Most preferably, photodynamic therapy is administered about one to about two weeks subsequent to the initiation of hormonal rhythm adjustment treatment.

In mouse models, the typical response observed using PDT alone is that tumor “cure” (tumor-free for at least 90 days) of 4-8 mm diameter tumors can be achieved in a majority of the cases, largely dependent upon the tumor size at the time of PDT. Treatment of tumors consisting of the adjustment of only prolactin daily rhythms, as described in U.S. patent application Ser. No. 08/271,881 filed Jun. 7, 1995, caused significant decreases in the growth of tumor tissue. Complete eradication of tumors, though, were not routinely achieved. It was unexpectedly discovered, however, that if the administration of prolactin, at the appropriate time interval, was combined with PDT treatment then the tumor cure rate was close to 100%. This level of response has not been heretofore obtainable by using either PDT or prolactin resetting therapy alone. Thus a combination PDT and the adjustment of prolactin daily rhythms show a synergistic effect in reducing the growth rate of or eradicating tumors. This synergistic effect is entirely unexpected, because there is no teaching in the prior art nor any reason for one of ordinary skill in the art to expect that prolactin would have any enhancing effects on the type I and type II photoreactions in which these dyes participate. Similarly, there is no teaching or suggestion in the prior art that would lead one to expect that timed administration of melatonin would act synergistically with PDT to kill tumors.

The present invention is further described and will be better understood by referring to the working Examples set forth below. These non-limiting Examples are to be considered illustrative only of the principles of the invention. Further, since numerous modifications and changes will readily occur to those skilled in the art, it is not desired to limit the invention to the exact construction and operation shown and described. Accordingly, all suitable modifications and equivalents may be used and will fall within the scope of the invention and the appended claims.

### EXAMPLE 1

### Combination Photodynamic Therapy Plus Neuroendocrine Resetting Therapy to Inhibit Tumor Growth

Adult (6-7 wk old) male Balbic mice were subcutaneously injected with EMT-6 cells derived from a murine mammary sarcoma (1.7×10⁶ cells) on the hind quarter and were divided into four groups (n=10 per group) and treated as follows: Group 1 (D+L) received photodynamic therapy at 14 days after tumor inoculation. Photodynamic therapy consisted of subcutaneously injecting a benzophenothiazine photosensitizer (ETNBS, FIG. 7)(7.5 mg/kg of body weight) and 3 hrs later irradiating the tumor with 652 nm light at a power density of 150 mW/cm² and a total energy of 100 J/cm². Group 2 (PRL) received intraperitoneal ovine prolactin (20 mcg/mouse) at 10 hours after light onset starting at 7 days after tumor inoculation and continuing for 14 days. Group 3 (D+L+PRL) received both PDT (as described above) and prolactin (as described above). Group 4 (CON) remained untreated (control). Fourteen days following PDT tumor volume was determined in all animals. The results are shown in FIG. 5.

### EXAMPLE 2

Example 1 was repeated with different PDT power density and energy characteristics (for groups 2 and 3) which better optimize the PDT effect. The power density was changed to 50 mW/cm² and the total energy was increased to 180 J. Such changes have been shown to dramatically increase the PDT effect, theoretically by allowing for better reoxygenation of the tumor tissue which 1) increases the type II dependent PDT effect and 2) re-oxidizes reduced dye to the “active” form again, increasing the PDT effect. Under these conditions, tumor “cure” (tumor-free for at least 90 days) of 4-8 mm diameter tumors can be achieved in 70-100% of the cases largely dependent upon the tumor size at the time of PDT. However, if intraperitoneal prolactin administration (20 mcg/mouse/day at 10 hours after light onset) is added to PDT treatment (starting from the day of tumor cell inoculation), then the tumor cure rate is 100%.

The typical response observed using PDT alone with the PDT parameters employed are (in sequence): 1) an inflammation of the photoirradiated spot encompassing the tumor within about 30 minutes which increases to a maximum inflammation at about 3-5 hours; 2) inflammation completely subsides in 48-72 hours leaving noticeable tumor which takes 14 days to regress completely. Eschar formation occurs at 24-48 hours.

Contrariwise, when intraperitoneal prolactin injections are added to the PDT as described above, 100% of the treated animals exhibit severe eschar formation within 24 hours and complete tumor eradication occurs within this time frame. At 4-7 days the irradiated area has healed completely. This response cannot be obtained with PDT or prolactin alone.

### EXAMPLE 3

Adult (6-7 wk old) male Balb/c mice are subcutaneously injected with EMT-6 cells derived from a murine mammary sarcoma (1.7×10⁶ cells) on the hind quarter and are divided into four groups (n=10 per group) and are treated as follows: group 1 receives photodynamic therapy (PDT) at 14 days after tumor inoculation. PDT consists of intravenously injecting a benzophenothiazine photosensitizer (Dye 4-115)(2.5 mg/kg of body weight) and 1 hr later irradiating the tumor with a broad wavelength light source (600-700 nm) at a power density of 50 mW/cm² and a total energy of 100 J/cm². Group 2 receives intraperitoneal ovine prolactin (20 mcg/mouse) at 10 hours after light onset starting at 7 days after tumor inoculation and continuing for 14 days. Group 3 receives both PDT (as described above) and prolactin (as described above). Group 4 remains untreated (control). Fourteen days following PDT tumor volume is determined in all animals.

The typical response which is observed using PDT alone with the PDT parameters employed are (in sequence): 1) an inflammation of the photoirradiated spot encompassing the tumor within about 30 minutes which increases to a maximum inflammation at about 3-5 hours; 2) inflammation completely subsides in 48-72 hours leaving noticeable tumor which takes 14 days to regress completely. Eschar formation occurs at 24-48 hours.

Contrariwise, when intraperitoneal prolactin injections are added to the PDT as described above, 100% of the treated animals will exhibit severe eschar formation within 24 hours and complete tumor eradication occurs within this time frame. At 4-7 days the irradiated area will heal completely. This response cannot be obtained with PDT or prolactin alone.

EXAMPLE 4

### Combination Photodynamic Therapy Plus Neuroendocrine Resetting Therapy with Bromocriptine to Inhibit Tumor Growth

Adult (6-7 wk old) male Balb/c mice are subcutaneously injected with EMT-6 cells (1.7×10⁶ cells) on the hind quarter and are divided into four groups (n=10 per group) and are treated as follows: group 1 receives photodynamic therapy (PDT) at 14 days after tumor inoculation. PDT consists of subcutaneously injecting a benzophenothiazine photosensitizer (EtNBS)(7.5 mg/kg of body weight) and 3 hrs later irradiating the tumor with 652 nm light at a power density of 50 mW/cm² and a total energy of 100 J/cm². Group 2 receives intraperitoneal bromocriptine (50 mcg/mouse) at 0 hours after light onset starting at 7 days after tumor inoculation and continuing for 14 days. Group 3 receives both PDT (as described above) and intraperitoneal bromocriptine (as described above). Group 4 remains untreated (control). Fourteen days following PDT tumor volume is determined in all animals. Tumors are found to be significantly reduced in the mice receiving either PDT treatment or intraperitoneal bromocriptine treatment, and are found to be significantly reduced or entirely eradicated in the mice receiving both therapies.

### EXAMPLE 5

### Combination Photodynamic Therapy Plus Neuroendocrine Resetting Therapy with Melatonin to Inhibit Tumor Growth

Adult (6-7 wk old) male Balb/c mice are injected subcutaneously with EMT-6 cells (1.7×10⁶ cells) on the hind quarter and are divided into four groups (n=10 per group) and are treated as follows: group 1 receives photodynamic therapy (PDT) at 14 days after tumor inoculation. PDT consists of subcutaneously injecting a benzophenothiazine photosensitizer (EtNBS)(7.5 mg/kg of body weight) and 3 hrs later irradiating the tumor with 652 nm light at a power density of 50 mW/cm² and a total energy of 100 J/cm². Group 2 receives intraperitoneal melatonin (8 mg/kg) at 10 hours after light onset starting at 7 days after tumor inoculation and continuing for 14 days. Group 3 receives both PDT (as described above) and intraperitoneal melatonin (as described above). Group 4 remains untreated (control). Fourteen days following PDT tumor volume is determined in all animals. Tumors are found to be significantly reduced in the mice receiving either PDT treatment or intraperitoneal melatonin treatment, and are found to be significantly reduced or entirely eradicated in the mice receiving both therapies.

### EXAMPLE 6

### Combination Photodynamic Therapy Plus Neuroendocrine Resetting Therapy with Bromocriptine and Melatonin to Inhibit Tumor Growth

Adult (6-7 wk old) male Balb/c mice are subcutaneously injected with EMT-6 cells (1.7×10⁶ cells) on the hind quarter and are divided into four groups (n=10 per group) and are treated as follows: group 1 receives photodynamic therapy (PDT) at 14 days after tumor inoculation. PDT consists of subcutaneously injecting a benzophenothiazine photosensitizer (EtNBS)(7.5 mg/kg of body weight) and 3 hrs later irradiating the tumor with 652 nm light at a power density of 50 mW/cm² and a total energy of 100 J/cm². Group 2 receives intraperitoneal melatonin (8 mg/kg) at 10 hours after light onset and intraperitoneal bromocriptine (50 mcg/mouse) at 0 hours after light onset starting at 7 days after tumor inoculation and continuing for 14 days. Group 3 receives both PDT (as described above) and intraperitoneal bromocriptine and intraperitoneal melatonin (as described above). Group 4 remains untreated (control). Fourteen days following PDT tumor volume is determined in all animals. Tumors are found to be significantly reduced in the mice receiving either PDT treatment or bromocriptine and melatonin treatment, and are found to be significantly reduced or entirely eradicated in the mice receiving both therapies.

What is claimed is:

1. A method for treating a mammal bearing one or more tumors, said mammal having a prolactin and a melatonin daily rhythm and in need of such treatment comprising the steps of:
comparing the prolactin profile of said tumor bearing mammal to a normal prolactin profile for healthy mammals of the same species and sex;
adjusting the prolactin profile of the mammal by administering prolactin enhancers or reducers in order that said prolactin profile conforms to or approaches the normal prolactin profile for healthy members of the same species and sex of said mammal;
contacting the cells of said tumor with a photosensitizer; and
exposing said contacted tumor cells to light of a predetermined wavelength and power density and energy level.

2. The method of claim 1 wherein said comparing step further comprises measuring the blood prolactin level of said tumor bearing mammal at spaced apart intervals within a 24-hour period to generate a prolactin profile for said mammal.

3. The method of claim 2 wherein said comparing step reveals that said tumor bearing mammal has (i) blood prolactin levels lower than 1 standard error of the mean (SEM) be low the normal night time prolactin level at two spaced apart time intervals or (ii) a blood prolactin level lower than 2 SEM below the normal night time prolactin level at one time point; and
said adjusting step comprises administering to said tumor bearing mammal a prolactin enhancer at a predetermined time or times to increase the night time prolactin levels of said mammal so that said mammal's night time prolactin level conforms to or approaches the normal night time prolactin profile.

4. The method of claim 3 wherein said prolactin enhancer is a member selected from the group consisting of melatonin, metoclopramide, domperidone and 5-hydroxytryptophan.

5. The method of claim 3 wherein said tumor bearing mammal is a human.

6. The method of claim 5 wherein said prolactin enhancer is melatonin and said melatonin is administered in amount within the range of 0.5-20 mg/person/day.

7. The method of claim 1 wherein said adjustment is continued until the prolactin rhythm of said mammal is reset to conform to or approach the normal prolactin profile and continues in its reset condition after cessation of said adjustment.

8. The method of claim 2 wherein said adjusting step comprises administering to said tumor bearing mammal a prolactin reducer at a predetermined time or times to decrease the day time prolactin levels of said mammal so that said mammal's day time prolactin level conforms to or approaches the normal night time prolactin profile.

9. The method of claim 8 wherein said mammal is a human, said prolactin reducer is bromocriptine, said bromocriptine is administered in an amount within the range of 0.2 to 8.0 mg/person/day, and said predetermined time is between about 6:00 h and 10:00 h.

10. The method of claim 9 wherein said bromocriptine is administered in an amount within the range of 0.8 to 4.8 mg/person/day.

11. The method of claim 10 wherein said bromocriptine amount is within the range of 0.8-3.2 mg/person/day.

12. The method of claim 6 wherein said predetermined time is about bedtime.

13. The method of claim 1 wherein said photosensitizer:
is positively charged;
is sufficiently lipophilic to be taken up by said tumor;
is retained substantially longer in the cells of said tumor than in non-tumor cells;
has a high absorption coefficient in the 600-900 nm light spectral region;
is capable of sensitizing tumor cells to killing by light exposure; and
is administered in an effective amount to sensitize said tumor to light.

14. The method of claim 13 wherein said photosensitizer is selected from the group consisting of porphyrin dyes, phthalocyanine dyes, cyanine dyes, benzophenoxazine analogs, and pharmaceutically acceptable salts thereof.

15. The method of claim 13 wherein said prolactin reducer or enhancer is administered orally, by injection, transdermally, or intranasally.

16. The method of claim 13 wherein said photosensitizer is administered intravenously, intraperitoneally, subcutaneously, or intralesionally.

17. The method of claim 16 wherein said effective amount of said photosensitizer is between about 0.1 and 15 mg/kg of body weight.

18. The method of claim 16 wherein said effective amount is between about 0.5 and 10 mg/kg of body weight.

19. The method of claim 18 wherein:
the energy level of said light is between about 5 and 400 Joules/cm²;
the power density of said light is between about 50 and 200 mWatts/cm²;
said tumor cells are exposed to said light between about 0.5 and 8 hours after administration of said photosensitizer.

20. The method of claim 16 wherein said effective amount is about 5.0 mg/kg of body weight.

21. The method of claim 19 wherein:
the energy level of said light is about 100 Joules/cm²;
the power density of said light is about 50 mWatts/cm²;
said tumor cells are exposed to said light at about 1 hour after administration of said photosensitizer.

22. The method of claim 19 wherein said photosensitizer is a benzophenothiazine.

23. The method of claim 21 wherein said photosensitizer is a benzophenothiazine.

24. The method of claim 22 wherein said benzophenothiazine is a member selected from the group consisting of Dye 4-115 and 5-ethylamino-9-diethylamino-benzo[a]phenothiazinium chloride.

25. The method of claim 23 wherein said benzophenothiazine is a member selected from the group consisting of Dye 4-115 and 5-ethylamino-9-diethylamino-benzo[a]phenothiazinium chloride.

26. The method of claim 13 wherein said contacting and exposing steps occur between about 7 and 14 days after initiation of said adjusting step.

27. The method of claim 15 wherein:
said mammal is a human;
said prolactin reducer is bromocriptine;and
said prolactin enhancer is selected from the group consisting of prolactin, melatonin, metoclopramide, domperidone, and 5-hydroxytryptophan.

28. The method of claim 27 wherein said prolactin enhancer is selected from the group consisting of prolactin and melatonin.

29. The method of claim 27 wherein said bromocriptine is administered at a time between about 6:00 h and 10:00 h and in an amount between about 0.8 and 8.0 mg/person/day.

30. The method of claim 28 wherein said prolactin enhancer is melatonin and wherein said melatonin is administered at about bedtime and in an amount between about 0.5 and 20 mg/person/day.

31. The method of claim 29 wherein:
the energy level of said light is about 100 Joules/cm²;
the power density of said light is about 50 mWatts/cm²;
said tumor cells are exposed to said light at between about 1 and about 3 hours after administration of said photosensitizer;
said photosensitizer is a member selected from the group consisting of Dye 4-115 and 5-ethylamino-9-diethylamino-benzo[a]phenothiazinium chloride; and
said photosensitizer is administered in an amount between about 1 and 5 mg/kg of body weight.

32. The method of claim 30 wherein:
the energy level of said light is about 100 Joules/cm²;
the power density of said light is about 50 mWatts/cm²;
said tumor cells are exposed to said light at between about 1 and about 3 hours after administration of said photosensitizer;
said photosensitizer is a member selected from the group consisting of Dye 4-115 and 5-ethylamino-9-diethylamino-benzo[a]phenothiazinium chloride; and
said photosensitizer is administered in an amount between about 1 and 5 mg/kg of body weight.

33. A method for treating a mammal bearing one or more tumors, said mammal having prolactin and melatonin daily rhythms and in need of such treatment comprising the steps of:
adjusting the prolactin and melatonin profiles of the mammal by administering prolactin enhancers or reducers and melatonin in order that said prolactin and melatonin profiles conform to or approach the corresponding normal profiles for healthy members of the same species and sex of said mammal;
contacting the cells of said tumor with a photosensitizer; and
exposing said contacted tumor cells to light of a predetermined wavelength and power density and energy level.
